(19) United States
(12) Patent Application Publication
(10) Pub. No.: US 2010/0228160 A1
(43) Pub. Date: Sep. 9, 2010

(54) APPARATUS FOR ACTIVITY MONITORING

(75) Inventor: Thomas Schweizer, Ebersberg (DE)

Correspondence Address: OCCHIUTI ROHLICEK & TSAO, LLP, 10 FAWCETT STREET, CAMBRIDGE, MA, 02138, US

(73) Assignee: Aipermon GmbH & Co. KG, Munchen (DE)

(21) Appl. No.: 12/540,715

(22) Filed: Aug. 13, 2009

(30) Foreign Application Priority Data
Mar. 3, 2009 (DE) 20 2009 003 034.2

(51) Int. Cl. A61B 5/11

(52) U.S. Cl. 600/595

(57) ABSTRACT

The application relates to a system for monitoring an energy balance of a subject, a portable monitoring apparatus to be used as a component of such a system, and an adequate computer program product. The system for monitoring an energy balance of a subject comprises an individual data input means, an individual data storing means, a basal metabolic rate calculating means, a food data input means, a nutritional value data base, a nutritional value calculating means, a movement data detecting means, an active metabolic rate calculating means, an energy balance calculating means, and an energy balance output means.

This invention relates to a system for monitoring an energy balance of a subject respectively a person, a portable monitoring apparatus to be used as a component of such a system, and an adequate computer program product.

Overweight is a problem to be taken seriously in many countries. Overweight may lead to a number of secondary diseases.

Causes for overweight may be changed dietary habits, such as higher calorie food intake on the one hand, and lack of movement on the other. Weight loss programs are offered by various organizations and companies, focusing on dietary and physical activity programs aiming to improve the ratio between caloric intake and caloric expenditure.

Scientific studies have shown that a long-term weight loss has to be linked to a change of behavior, in particular controlled food intake and sufficient physical activity.

Some measuring devices have been known which aim to determine a preferably accurate caloric balance from caloric intake and caloric expenditure. The caloric expenditure rate is derived from the basal metabolic rate of a living entity and the number of calories expended by physical activity.

There have been several approaches to provide systems that can be helpful for users to influence their personal caloric balance.

U.S. Pat. No. 6,571,200 gives a survey of various possibilities. In this publication it is described how caloric expenditure may be determined rather accurately by caloric expenditure measurements. It describes the use of measurements by indirect calorimetry, by measuring the physical activity, and inputting the number of calories taken in at meals by way of inputting the kind and quantity of food eaten.

The publication U.S. Pat. No. 6,095,949 describes how, in addition to measuring the physical activity, the number of calories taken in at meals is determined by accessing a data base.

In DE 197 42 153 it is described how to convert a taken meal into an input. Based on the physical activity, a caloric balance is calculated.

The systems mentioned above are partly lacking in accuracy and difficult to handle in practice. Users of these systems are often overwhelmed by handling them, which may cause them to stop using such systems after an initial familiarization period. Thus, such systems are only suitable with limitations to provide the desired help to users.

The object of the present invention is to provide a system for monitoring the energy balance of a subject, a portable monitoring apparatus to be used as a component of such a system, and a method ensuring simple handling while providing an accurate determination of the subject's energy balance.

This object is solved by the subject-matter of the independent claims and the aspects described hereinafter. Preferred embodiments are the subject-matter of the dependent sub-claims and may be derived from the following description.

A first aspect relates to a system for monitoring an energy balance of a subject, comprising:

an individual data input means designed to input subject-related individual data into the system,
an individual data storing means designed to store the inputted individual data in the system,
a basal metabolic rate calculating means designed to calculate a subject-related basal metabolic rate on the basis of the individual data, and provide it in the system,
a food data input means designed to facilitate respectively allow the input of food data into the system,
a nutritional value data base having nutritional values assigned to the food data,
a nutritional value calculating means designed to calculate an overall nutritional value on the basis of the inputted food data and the nutritional values assigned in the nutritional data base,
a movement data detecting means designed to be operatively arranged on the subject and to detect movements and/or movement changes of the subject, and to provide corresponding movement data,
an active metabolic rate calculating means designed to calculate an active metabolic rate on the basis of the detected movement data,
an energy balance calculating means designed to calculate an energy balance on the basis of the active metabolic rate, the basal metabolic rate and the nutritional value, and energy balance output means for outputting the determined energy balance.

The term ‘energy balance of a subject’ denotes a balance to be determined, e.g., for a certain period of time, between a food metabolic rate due to food intake by a subject, and the energy expended by the subject, e.g., based on a basal metabolic rate and an active metabolic rate. Both the basal metabolic rate and the active metabolic rate are calculated approximatively on the basis of the underlying data. Basal metabolic rate denotes an approximation of an energy amount required by a subject per unit time when completely at rest. Active metabolic rate denotes an approximation of an energy amount required by a subject due to activity, in particular physical activity, in addition to the basal metabolic rate. The active metabolic rate may also be called ‘performance metabolic rate’. A negative balance indicates that more energy has been expended than taken in within a given period of time. In this case, by displaying the negative balance it is indicated to the subject that he or she is losing weight. In case of a positive balance, it will be indicated to the subject that he or she is gaining weight.

The system may be completely accommodated in a portable monitoring apparatus, or it may comprise several units, such as a portable monitoring apparatus, a stationary computer and/or a server on the internet. In the following, the term ‘portable monitoring apparatus’ is also partly designated as a monitoring apparatus. The term ‘monitoring apparatus’ in this sense denotes an apparatus designed with regard to its size and weight in such a way that the subject may carry it without feeling it to be an unreasonable encumbrance. A monitoring apparatus may have a weight of less than 500 g, in particular a weight of less than 250 g or less than 200 g. The term ‘computer’ is used below in the sense of a stationary computer. It refers to a computer that the subject would feel to be too big or too heavy to carry continuously. This term, therefore, also comprises so-called laptops, notebooks, or netbooks. Computers may have dimensions with at least one lateral length greater than 15 cm, or weighing more than 500 g.

The individual data input means is designed to input subject-related individual data into the system and, respectively, to enable a subject to input individual data into the system. For the purpose of inputting, the individual data input means may comprise a data transfer module providing an interface between an external device, such as a computer, and a portable monitoring apparatus. The interface may be provided with an associated interface protocol and designed to allow a data-exchanging connection (signal connection) between the monitoring apparatus and the computer so that individual data may be transferred to the monitoring apparatus via the signal connection. The individual data may comprise an externally calculated basal metabolic rate, as will be described below.

The individual data input means may, e.g., comprise a computer keyboard connected with a stationary computer. By using such individual data input means the user may enter his personal individual data into the computer, e.g., in a menu-controlled manner. On the basis of the inputted individual data, a subject-related basal metabolic rate may be calculated in the system, e.g., on the computer, or on the portable monitoring apparatus. It is further conceivable for the individual data input means to be arranged as a component of the portable monitoring apparatus, designed to facilitate input of the individual data directly into the portable monitoring apparatus. Individual data input means of a monitoring apparatus may, e.g., comprise a combination of touch-sensitive operating elements, such as keys, linked to a selection menu, and a display means, such as a display. The touch-sensitive operating elements may, e.g., be arranged as so-called softkeys, providing various context-related input functions that may be shown to the subject on the display. Person-related individual data may be, e.g., the subject's age and/or the subject's gender and/or the subject's weight and/or the subject's size. Individual data may also be body fat percentage, muscle mass, a customary activity pattern of the subject, or other data that may serve for calculating a basal metabolic rate and/or for calculating an active metabolic rate and/or for categorizing or individualizing the subject's food intake. Individual data relating to an activity pattern of the subject may, e.g., comprise one or more of the following categories the subject usually pursues: hard physical labor, light physical work, office work, standing work, etc. The individual data storing means may be provided, e.g., as a separate memory chip or as a memory area of a memory chip, such as a non-permanent memory chip, but in particular a permanent re-writable memory chip.

The basal metabolic rate calculating means may be provided, e.g., by an external processor such as a computer CPU, or by a processor of the monitoring apparatus. Such a processor may recalculate the subject-related basal metabolic rate from case to case, if needed, on the basis of the inputted individual data so that the basal metabolic rate need not be stored as a distinct value. It is also conceivable to calculate the basal metabolic rate on the basis of the inputted individual data and store it together with the other individual data as a separate value. The food data input means may be designed to enable a subject to input the food data directly into the portable monitoring apparatus. Food data may comprise information about meals and/or the quantity and/or quality of certain foods. The food data input means may be designed to facilitate input of food data into the system by means of individualized categories. The term ‘by means of individualized categories’ refers to a modifiability of the food data input means on the basis of typical dietary habits of the respective subject. ‘Category’ means that the subject is offered a choice of certain categories of food intake for entry, such as various meals the subject typically eats on a regular basis. ‘Individualized’ means that, e.g., the kind and/or number of categories for choice are adapted to the information given by the subject based on a request of the subject, and are thus available in an individualized manner for input. Individualized categories may comprise, e.g., such categories as ‘breakfast’, ‘first snack’, ‘lunch’, ‘second snack’, and/or ‘dinner’. In addition, the food data input means may be designed so as to enable the subject to input a concrete number of calories, if known. The food data input means may comprise keys provided on the portable monitoring apparatus and/or other operating elements that may be operated by the subject. It is also possible to use voice-operated input. The food data input means may further comprise display means. In operation, menu items may be provided to the subject via the display means, from which the subject may make a suitable choice by utilizing the operating elements.

With reference to certain food data that may be entered into the system, the nutritional value data base provides nutritional values assigned to these food data. Nutritional values are energy quantities that may, e.g., be given as kilocalories or kilojoules. Nutritional values provided by the nutritional value data base may, e.g., be food-related, i.e., given as a specific nutritional value of a certain foodstuff, and/or category-related, i.e., as an absolute nutritional value of a categorized unit such as a meal. Category-related nutritional value data may be provided by individualized programming in the nutritional value data base, e.g., with reference to an individual's diet. Nutritional values, in particular those linked with an individualized category of the food data input means, may thus be provided in an individually programmed fashion.

On the basis of the inputted food data and/or the provided nutritional values, the overall nutritional value ingested by a subject, e.g., at a meal, may be determined by means of a nutritional value calculating means. Such a calculation may comprise retrieving one or more nutritional values, corresponding to the food data input, from the nutritional value data base. Further, specific nutritional values of a foodstuff may be multiplied by the entered quantity of the foodstuff. One or more individual nutritional values provided in this manner may be added up to give an overall nutritional value, which overall nutritional value may be determined on an event-related basis, i.e. per meal, or on a time-related basis. Over a certain operating period (such as a day, a week, a month, etc.) several overall nutritional values may be determined, which may be balanced one after another.

The movement data detecting means may be provided, e.g., as a component of a portable monitoring apparatus. The monitoring apparatus may be carried by the subject, and thus the movement data detecting means is arranged on the subject when the movement data detecting means are in operating mode, and may detect movements and/or movement changes of the subject. An active energy expenditure, or a time-related active (performance') metabolic rate, i.e. an energy amount, may be calculated directly from the movement data. It is also conceivable to provide the movement data in such a way that conclusions may be drawn for a movement pattern on the basis of movement data. Such movement patterns may be determined, e.g., by comparing the movement data with movement patterns provided in a movement pattern data base. On the basis of such movement patterns an active energy expenditure or an active (‘performance’) metabolic rate may again be determined. The movement data detecting means may comprise, e.g., one or more movement sensors such as an acceleration sensor, in particular a three-dimensional acceleration sensor such as a three-axis acceleration sensor. Further, GPS assemblies may be used. It is also conceivable to use mechanical elements as they are known from pedometer models, or any other movement sensors. Movements and/or movement changes may be detected and/or recorded on a time basis, i.e., together with information on the exact time and duration of a movement and/or a movement change.

On the basis of movement data from the movement data detecting means and, optionally, from the entered individual data, such as weight, size and/or age, the active metabolic rate may be determined by means of the active metabolic rate calculating means, i.e., the energy expended in correlation with a certain movement or movement pattern. Such a calculation may comprise determining a movement pattern from the movement pattern data base and/or retrieving a specific rating of a determined movement pattern from the movement pattern data base and/or a user-controlled input of a physical activity. The active metabolic rate may be determined on a time basis. Over a certain operating period (such as a day, a week, a month, etc.), several active metabolic rates may be determined, added up and balanced, e.g., one after another.

The basal metabolic rate, active metabolic rate and nutritional values may be balanced successively by means of the energy balance calculating means during an operating period and/or they may be balanced at the end of the operating period; a difference between energy input and energy consumption may be determined. The nutritional value calculating means, the active metabolic rate calculating means, the energy balance calculating means, the basal metabolic rate calculating means and other designated calculating means may be provided, e.g., in the form of one or more external processors such as a CPU of a computer, or by one or more processors of the monitoring apparatus. It is also conceivable to calculate the basal metabolic rate externally, e.g., on the computer, and to execute the other calculations in the monitoring apparatus.

The energy balance output means may be provided, e.g., by optical and/or acoustical output means. The output means may comprise, e.g., a display that allows for representing a negative or positive balance by a symbol, and/or indicating the balance value, i.e. the difference between energy input and energy expenditure, as an amount by a numeric value. The subject may also be shown separate values for, e.g., basal metabolic rate, active metabolic rate and/or nutritional value intake. The basal metabolic rate may, e.g., be displayed as an ascending line over the time passed within a period of use, so that the subject may, e.g., be given an indication of the overall energy expenditure accumulated over the time passed. In addition to an optical display, an acoustic warning signal and/or an acoustic success signal may be given when the nutritional value intake exceeds the summed up expenditure from the (accumulated) basal metabolic rate and active metabolic rate, or, respectively, when the summed up expenditure exceeds the nutritional intake. The warning signal and/or success signal may also be given optically. The system may be designed to be configurable so that the subject may choose from one or more of the possibilities mentioned. The interaction between man and machine may be improved by such a design. A motivating effect for the user may thus be enhanced.

One embodiment of the system described above relates to a system wherein the basal metabolic rate calculating means has a signal connection with a basal metabolic rate storing means designed to store the basal metabolic rate calculated by the basal metabolic rate calculating means. Such a signal connection may be provided, e.g., as a wireless signal connection between the computer and the portable monitoring apparatus. In this case, the basal metabolic rate may be calculated using the basal metabolic rate calculating means on the computer, and may be transferred via the signal connection to the monitoring apparatus, where it may be stored in the basal metabolic rate storing means. It is also conceivable to configure the basal metabolic rate calculating means as a component of the monitoring apparatus so that the signal connection is provided in the monitoring apparatus by appropriate conductor paths connecting the basal metabolic rate storing means with the basal metabolic rate calculating means.

Another embodiment of the system relates to a system, wherein the basal metabolic rate storing means has a signal connection with the energy balance calculating means, wherein the energy balance calculating means is designed to retrieve the basal metabolic rate stored in the basal metabolic rate storing means.

Another embodiment of the system described above relates to a system, wherein no basal metabolic rate storing means is provided and wherein the basal metabolic rate calculating means has a signal connection with the individual data storing means, wherein the basal metabolic rate calculating means is designed to calculate the basal metabolic rate on the basis of the individual data stored in the system and provide the calculated basal metabolic rate to the energy balance calculating means. In this case, the basal metabolic rate calculating means may be identical with the energy balance calculating means and may be designed to concurrently calculate the basal metabolic rate in the course of the energy balance calculation. It is also conceivable that the basal metabolic rate calculating means first calculates the basal metabolic rate, and has a signal connection with the energy balance calculating means, so it can make the calculated basal metabolic rate available to the energy balance calculating means via the signal connection. According to this embodiment, the basal metabolic rate calculating means may be a component of the portable monitoring apparatus. An advantage of this embodiment is that a memory chip, or memory area, respectively, may be saved. Data relating to the basal metabolic rate need not be retrieved first, but may be calculated directly in the course of calculating, e.g., an energy balance. Further, in this embodiment, changes of individual data upon which the calculation of the basal metabolic rate is based, can be taken into account more easily.

An embodiment according to any of the systems described above relates to a system, wherein the food data input means comprises a menu display means and a menu selection means, wherein the menu display means is designed to indicate to the subject a categorized selection menu, wherein the selection menu preferably has a tree structure showing several menu layers and several entries per menu layer, and is designed to enable the user to enter the food data via the tree structure. According to this embodiment, the interaction between man and machine may be simplified. The term ‘categorized selection menu’ means that typical circumstances are offered for choice in a simplified and summarized manner. For example, the subject does not have to enter different foods and their quantities or distinct nutritional values into the apparatus, but rather may choose from various categories representing typified situations. Thus, the subject has the possibility to choose a suitable item from a number of typified items, each with an assigned nutritional value, which would correspond to the actual food intake. Thus, the subject is spared having to choose from a food data base that may comprise several thousands, or even several ten thousands of entries and may, therefore, prove to be very complicated to use. Thus, a fatigue effect on the subject is avoided, which might otherwise result in giving up using the apparatus, so that its positive effect on the subject's dietary and movement behavior would be lost.

Surprisingly it has been found that slight deviations from the actual food intake, which go along with a categorized selection, may be disregarded in the long-term view of several weeks or even months, as these slight deviations cancel each other over an extended period of time. A categorized selection is, therefore, sufficiently accurate and provides a significant simplification to the subject making the selection. For example, a selection menu may be designed to offer a subject at a first menu layer a choice among categories such as ‘breakfast’, ‘first snack’, ‘lunch’, ‘second snack’, ‘dinner’, and ‘direct calorie input’. If the subject chooses, e.g., the menu item ‘lunch’, they may then be offered, at a second menu layer, a choice among the categories of ‘small’, ‘medium’, ‘large’, and ‘direct calorie input’. The items ‘small’, ‘medium’ and ‘large’ may be linked with certain nutritional values in a data base, corresponding, e.g. to statistically determined averages for these meals, or they may have been individualized according to the subject's dietary habits. A categorized selection menu may also be time-controlled. For example, at a certain time the user may be offered as a first choice the most appropriate meal to be chosen by the subject at the respective time of the day. If, for example, the subject wants to enter food data at 12:00 o'clock noon, they may directly be offered the menu items ‘lunch small’, ‘lunch medium’, ‘lunch large’, and ‘other meal’. Thus, the subject may choose from the three lunch categories or choose ‘other meal’ if, e.g., they had just taken the first snack only, and would then get to another selection menu.

Another embodiment of the system described above relates to a system, wherein the tree structure of the categorized selection menu comprises not more than 10 items per menu layer and/or wherein the tree structure comprises not more than 5 menu layers. By designing the tree structure in this way, the man-machine interaction may be further simplified. It will take the subject less time to enter the data into the apparatus. It is also conceivable to design the tree structure in such a manner that it does not comprise more than six items per menu layer and does not have more than four menu layers, thus further simplifying the input for the subject.

Still another embodiment relates to a system described above, wherein the food data input means is designed to enable the subject to input concrete food data in addition to the categorized selection menu, wherein the input of concrete food data particularly comprises the input of a specific nutritional value and/or a specific food and/or a specific quantity of food. This design enables subjects who wish to make very exact data entries to choose between the option of a fast, categorized selection and the option of a more elaborate, more accurate entry of food data.

According to another embodiment of such a system, the food data input means may be designed to be individualized by the subject. An individualization of the food data input means may improve the accuracy of the input while maintaining the advantages of the above-mentioned, menu-controlled food data input. In addition to the above-mentioned menu design examples a menu may, e.g., be designed in such a way that a subject will be offered at the second menu layer a selection amongst ‘small’, ‘medium’, ‘large’, ‘favorite meal’, and ‘direct number of calories’. The items ‘small’, ‘medium’ and ‘large’ may be stored with specific individualized nutritional values in a data base individualized according to the subject's dietary habits. The menu item ‘favorite food’ may contain a number of foods frequently consumed by the subject, for which the corresponding individualized nutritional values are stored in the nutritional value data base. Individualization may be effected, e.g. by means of inputting specific individual data. Individualization may be carried out directly on the monitoring apparatus or on a computer, e.g., before placing the monitoring apparatus into operation, and then transferred to the monitoring apparatus.

In one embodiment of such a system, individualization of the food data input means may relate to limiting the number of menu layers, limiting the number of menu items per menu layer, the name of menu items, and/or it may relate to the nutritional values assigned to specific menu items. Individualization may be carried out, e.g., by means of suitable individualization means directly on the portable monitoring apparatus. Alternatively or additionally, it is also conceivable to carry out individualization on the computer and subsequently transfer it to the portable monitoring apparatus. Individualization in such a manner may be carried out, e.g. before start-up of the monitoring apparatus. It is further conceivable that such an individualization may be modified later. For the purpose of individualization, e.g., a data base such as a food data base may be provided to simplify individualization. By means of the food data base, the subject is enabled to put together a number of typical meals on the basis of their dietary habits. By using the compiled meals that may, e.g., include typical foods and their food quantities, a nutritional value of such a typical meal may be calculated, and it may be provided as an individualized menu item by the food data input means. Such an individualized menu item may refer both to the categorized menu items, such as ‘lunch small’, ‘lunch medium’ or ‘lunch large’, and a list of the subject's favorite meals. Moreover, individualization enables the subject to delete or supplement certain menu items. Other possibilities of individualization are also conceivable.

In another embodiment the system may have a food data storing means designed to store food data entered via the food data input means, and/or associated nutritional values in the system. The food data storing means may be designed to store food data with regard to several meals of a day, a week, a month or any other desired period of time. The stored food data may be retrieved from the food data storing means at a later time, and the corresponding information may be displayed to the subject. The system may be designed so that the information may, e.g., be displayed on the monitoring apparatus and/or on the computer. Displaying on the computer may be effected, e.g., by presenting a graph, by means of which the subject may easily retrace their dietary behavior. The food data storing means may be provided in the form of a memory chip or in the form of a memory area of a memory chip, which memory chip may also be arranged for storing other data such as individual data, basal metabolic rate, the nutritional values data base, movement data, movement patterns, and/or a movement pattern data base, etc.

In such a system according to another embodiment, the system may comprise a movement pattern data base, having movement patterns stored in the movement pattern data base that may be linked with the detected movement data. The movement pattern data base may be designed to detect, by means of the information provided in the movement pattern data base and on the basis of the detected movement data, a specific movement pattern and/or a specific expenditure value assigned to the movement pattern. Movement patterns may be, e.g., active, passive, housework, garden work, slow walking, fast walking, jogging, and/or any other movement patterns. By means of the specific expenditure value and certain individual data, an individual expenditure value may be determined. The movement pattern data base may have a signal connection with the active metabolic rate calculating means. The movement pattern data base may be provided as a component of the monitoring apparatus and/or the computer.

According to another embodiment, the system may have an activity pattern data base, including absolute and/or specific activity expenditure values that may be linked with activity information entered by the subject and stored in the activity pattern data base. The activity data may be requested together with other individual data and may comprise, e.g., a conventional activity pattern. The activity pattern data base may be designed in such a way that a specific, typical, absolute expenditure value for the subject may be predicted on the basis of the activity expenditure values provided in the activity pattern data base and/or the individual data of the subject. Activity patterns may be, e.g. active, passive, housework, garden work, slow walking, fast walking, jogging, and/or any other activity patterns. The activity pattern data base may be designed as a component of the movement pattern data base, and/or may have a signal connection with the active metabolic rate calculating means. The activity pattern data base may be provided as a component of the monitoring apparatus and/or the computer.

In another embodiment of such a system, the system may have a movement information storing means designed to store movement data provided via the movement data detecting means, and/or movement patterns linked to the movement data, in the system, in particular in the monitoring apparatus. The movement information storing means may be designed to store movement information of a day, a week, a month, or any other desired period of time. The stored movement information may be retrieved from the movement information storing means at a later time, and the corresponding information may be displayed to the subject. The system may be designed so that the information may, e.g., be displayed on the monitoring apparatus and/or on the computer. Displaying on the computer may be effected, e.g., by presenting a graph, by means of which the subject may easily retrace their movement behavior. The movement information storing means may be provided in the form of a memory chip or in the form of a memory area of a memory chip, which memory chip may also be arranged for storing other data such as individual data, basal metabolic rate, the nutritional values data base, movement data, movement patterns, and/or a movement pattern data base, etc.

In yet another embodiment of such a system, the system may have a time detecting means designed to link a basal metabolic rate and/or a food data input and/or movement data and/or a movement pattern with time data. A time detecting means may be provided, e.g., in the form of a separate timer and/or as a component of a CPU comprising a timer. By means of the time detecting means, the point in time and/or the duration of certain events may be detected. Such events may particularly relate to food intake and/or movement behavior, e.g., detecting the time and/or duration of an activity level and/or an activity class. Further, the time detecting means may be designed to provide time information in such a way that, e.g., the energy balance may be displayed on a time basis. For example, the basal metabolic rate may be broken up into individual parts over a day so that the energy balance displayed to the subject takes into account only the relevant, proportionate basal metabolic rate accrued so far.

Another embodiment of such a system relates to a variation comprising a distance calculating means. By using the distance calculating means, e.g., a covered distance and/or a negotiated height may be detected and then incorporated into the calculation of an active metabolic rate. The distance may be calculated on the basis of movement data and/or on the basis of data provided, e.g., via a GPS module.

Such a system, according to another embodiment, may comprise a sensing means designed to detect a physiological parameter of a subject. Detected physiological parameters may be, e.g. the pulse, body temperature, breathing, or other parameters, which may be incorporated into the calculation of the basal metabolic rate and/or the active metabolic rate.

Another embodiment of such a system relates to a system comprising a set target input means designed to enable a subject to input a set target. By means of the set target input means it is possible to enter and check a set target with regard to a daily, weekly and/or monthly activity, weight gain or loss, food consumption, or other parameters.

Another system comprises, according to another embodiment, an activity input means designed to enable a subject to input physical activities. An activity input means may facilitate the input of information supplementing the data detected by the movement data detecting means. Thus, the subject may enter information into the system, in particular the portable monitoring apparatus, when the subject has engaged in physical activity without carrying the monitoring apparatus. This may be an advantage, e.g., if the monitoring apparatus is not watertight and sports like swimming are to be taken into account in calculating the active metabolic rate and the energy balance, respectively. By means of the weight change calculating means a theoretical target weight change may be calculated, which should have occurred on the basis of the food data input, the detected movement data, and the calculated basal metabolic rate. A corresponding theoretical target weight may be compared to a measured actual weight. If the target weight and the actual weight diverge, a correction factor may be computed, by means of which the individual basal metabolic rate and/or the nutritional values assigned to specific food data in the nutritional value data base may be corrected. If, for example, no weight loss is achieved within a period of time although the caloric balance is negative, the stored basal metabolic rate may be decreased and/or the calories assigned to individual meals may be increased. This may, e.g. be effected by summing up the caloric balances of the relevant period with the comparison of weight change.

Another embodiment relates to a system according to any of the systems described above, comprising a portable monitoring apparatus suitable to be carried by a subject, which monitoring apparatus comprises at least the following components:
the individual data storing means,
the food data input means,
the nutritional value data base,
the nutritional value calculating means,
the movement data detecting means,
the active metabolic rate calculating means,
the energy balance calculating means, and
the energy balance output means.

The term ‘portable monitoring apparatus’ denotes a device designed with regard to its size and weight in such a way that it may be carried by a subject without the subject feeling this to be an unreasonable encumbrance.

Such a system, according to another embodiment, may further comprise a computer and/or a balance and/or a blood pressure unit and/or a blood sugar meter and/or a body fat meter and/or a central server. The central server may, e.g., be a processor having a data exchanging connection with the computer and/or the monitoring apparatus via the internet. The central server may, e.g., be designed to carry out a detailed evaluation of the data received by the monitoring apparatus, and to transmit to the subject an individualized feedback. The individualized feedback may comprise some advice, e.g., on dietary behavior and/or movement behavior and/or proper operation of the monitoring apparatus. This feedback may be sent automatically by the central server, or it may be sent by support staff. It is also conceivable for the monitoring apparatus only to carry out a rough evaluation, while the computer does a fine evaluation upon receiving the recorded data. The balance may, e.g., be a body fat scale. Weight data and/or body fat information may be provided by the balance in the system via a signal connection. For example, the information may be transferred to the monitoring apparatus and/or the computer. By means of the weight information and/or the body fat information, the basal metabolic rate may be recalculated, and/or corresponding individual data, in particular a stored basal metabolic rate, may be corrected.

In a system as described above, according to another embodiment a signal connection may be arranged between the monitoring apparatus and the computer and/or between the monitoring system and the balance and/or between the balance and the computer, which may be a wired or a wireless signal connection. A wired signal connection may be provided via a conventional interface, such as a USB interface. For this purpose, the monitoring apparatus may comprise a wired interface module for establishing a wired signal connection. A wireless signal connection may be provided, e.g., in the form of a bluetooth connection or a WLAN connection. For this purpose, the monitoring apparatus may additionally or alternatively comprise a wireless interface module for establishing a wireless signal connection. The signal connection may be designed to allow a unidirectional or a bidirectional data exchange between the components. Besides the specifically mentioned signal connections, other signal connections between other components of the system may be provided.

Another embodiment of the systems mentioned above relates to a system arranged as a portable monitoring system suitable to be carried by a subject.

A second aspect relates to a computer program product, comprising program parts which, when loaded onto a processor and executed, are designed to execute the following steps for monitoring the energy balance of a subject:
requesting of individual data,
calculating a basal metabolic rate on the basis of individual data relating to the basal metabolic rate,
requesting of food data,
determining a nutritional value on the basis of the requested food data,
detecting of movement data,
calculating an active metabolic rate on the basis of the movement data and the individual data relating to the active metabolic rate,
calculating an energy balance on the basis of the basal metabolic rate, the active metabolic rate, and the nutritional value,
outputting of the calculated energy balance.

Such a computer program product may, e.g., be a data carrier such as a CD-ROM, a DVD, a floppy disk, a flash memory, a hard disk, or another form of embodied data.

Further steps to be executed upon loading and executing the computer program product on the processor may be derived from the explanations given above with regard to the system for monitoring an energy balance. Such a processor may be a portable monitoring apparatus or a computer. It is also conceivable to execute some parts of the program on a computer and other parts of the program on the monitoring apparatus.

A third aspect relates to a method for individualizing a portable monitoring apparatus, comprising the following steps:
requesting of individual data relating to a basal metabolic rate, and of individual data relating to the active metabolic rate,
storing the individual data relating to the active metabolic rate in the monitoring apparatus,
requesting of dietary habits,
categorizing the dietary habits with regard to the number and kind of meals usually taken daily by the subject,
determining individualized nutritional values with regard to the meals on the basis of the requested dietary habits,
individualizing the selection menu of a food data input means, and
storing the individualized selection menu in the monitoring apparatus.

According to an embodiment of the method, the step of individualizing may further comprise the following steps:
calculating a basal metabolic rate on the basis of the individual data relating to the basal metabolic rate,
stoing the basal metabolic rate in the monitoring apparatus.

According to another embodiment of the method, the step of individualizing may comprise the following steps:
requesting input from the subject with regard to activity data providing information about some movement behavior of the subject,
calculating the subject's active metabolic rate to be expected on the basis of the requested activity data, e.g., by retrieving an activity expenditure value from an activity pattern data base,
calculating the overall metabolic rate to be expected from the active metabolic rate and the basal metabolic rate,
calculating a distribution of the subject's expected overall caloric expenditure over the categorized individual meals,
individualizing the selection menu of the food data input means, wherein an average number of calories to be entered for individual meals corresponds to the proportion of the expected overall caloric expenditure distributed over individual meals,
storing the individualized selection menu in the monitoring apparatus.

A fourth aspect relates to a method for monitoring an energy balance of a subject, comprising the following steps:
providing a portable monitoring apparatus for monitoring the energy balance of a subject,
requesting of individual data relating to a basal metabolic rate, and of individual data relating to an active metabolic rate,
storing the individual data in the portable monitoring apparatus,
calculating a basal metabolic rate on the basis of the individual data relating to the basal metabolic rate,
requesting of food data,
determining a nutritional value on the basis of the requested food data,
detecting of movement data,
calculating an active metabolic rate on the basis of the movement data and the individual data relating to the active metabolic rate,
calculating an energy balance on the basis of the basal metabolic rate, the active metabolic rate, and the nutritional value,
outputting the calculated energy balance.

According to another embodiment of the method, the method may comprise the individualizing steps of a method according to the third aspect.

The methods described with reference to the third and fourth aspects may comprise providing a computer and/or other apparatus of a system described above with reference to the third aspect. The methods described may comprise further steps arising from the description of the first aspect. A distinct scope of protection shall be assigned to the methods mentioned as well as the described and claimed apparatus.

In the following, some embodiments of the apparatus and methods will be individually described in detail in an exemplary manner by means of the figures. In part, the individual embodiments described comprise features that are not absolutely necessary to execute the claimed subject-matters; however, they provide desirable characteristics for certain applications. Therefore, embodiments that do not comprise all the features of the embodiments described below should also be considered as coming under the technical teaching described in this publication. Moreover, to avoid unnecessary repetitions, certain features will only be described with reference to individual embodiments. Thus, it is pointed out that embodiments should not just be taken by themselves, but should also be considered in an overall view. By taking such an overall view the person skilled in the art will recognize that individual embodiments may also be modified by incorporating one or more features from other embodiments. It is pointed out that a systematic combination of individual embodiments with one or more features described with reference to other embodiments may be desirable and meaningful and should, therefore, be taken into consideration and should also be regarded as being comprised by the description.

The figures show:

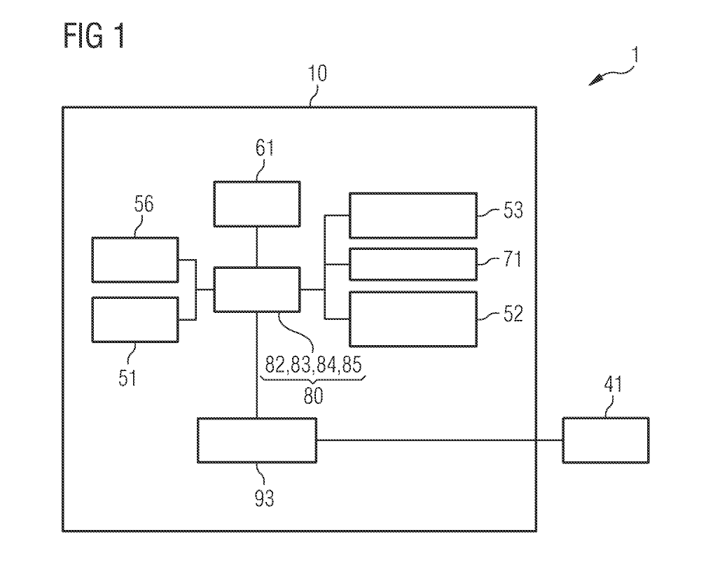

FIG. 1: a first embodiment of a system for monitoring an energy balance,

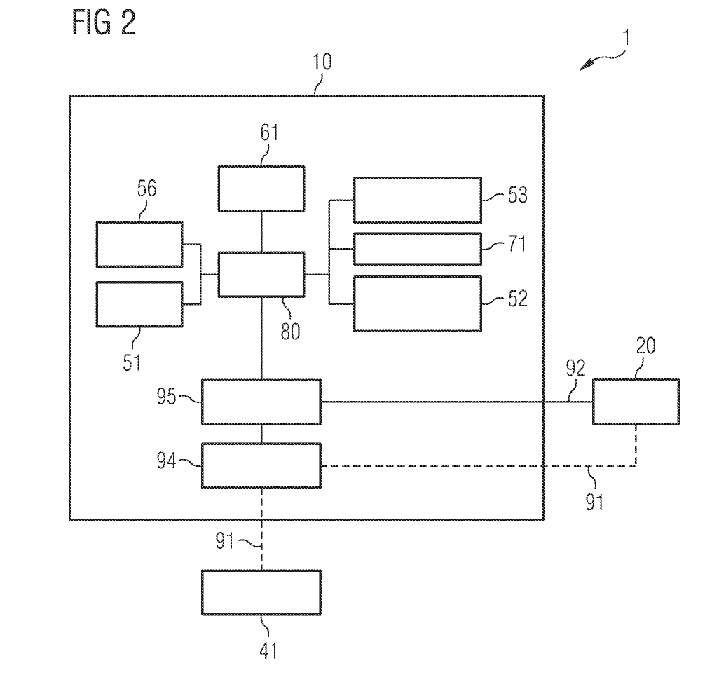

FIG. 2: a second embodiment of a system for monitoring an energy balance,

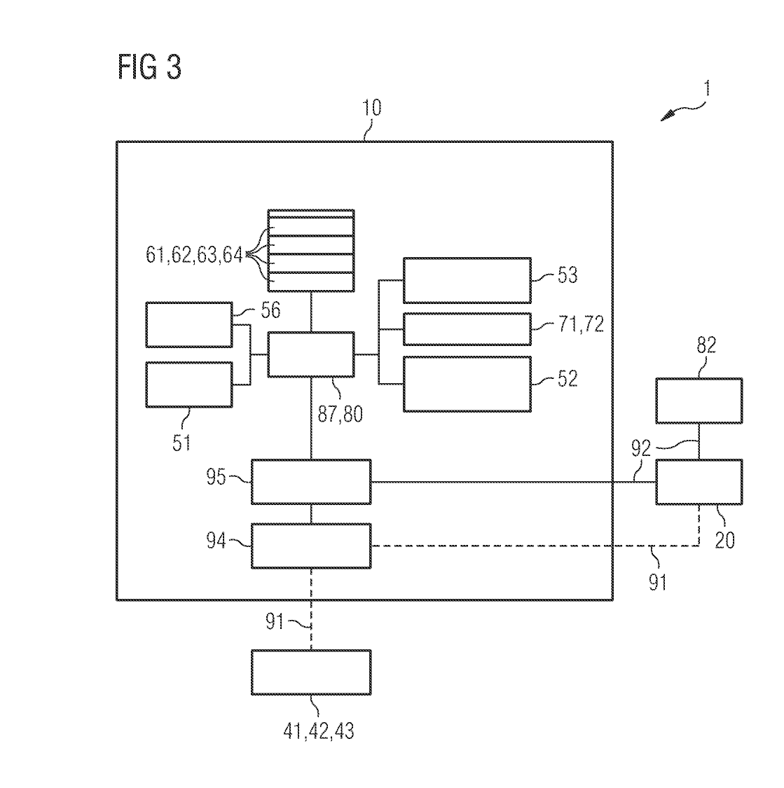

FIG. 3: a third embodiment of a system for monitoring an energy balance,

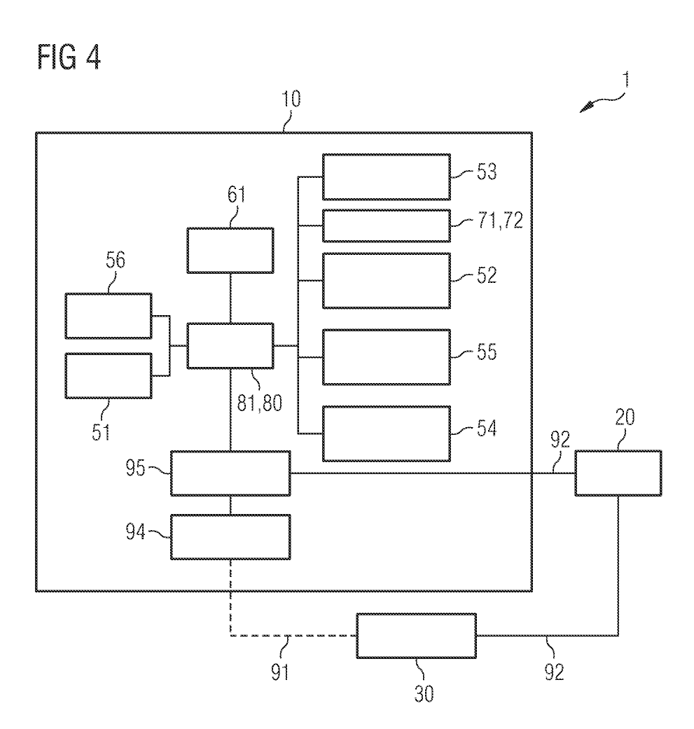

FIG. 4: a fourth embodiment of a system for monitoring an energy balance,

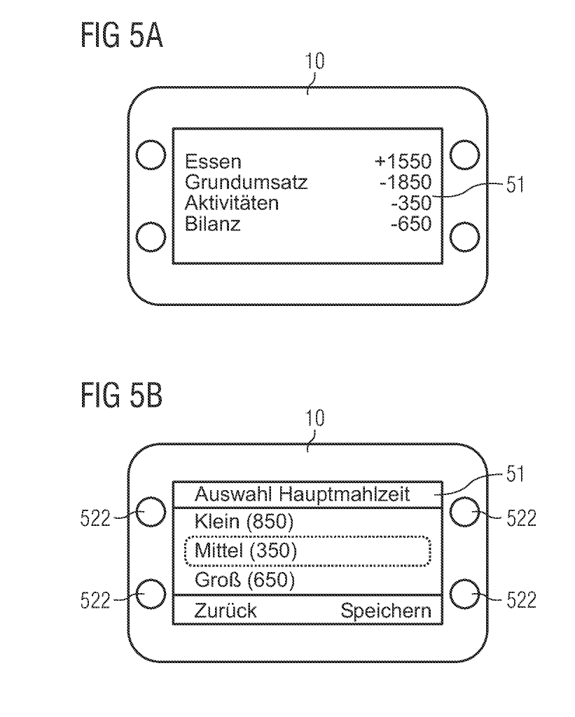

FIGS. 5a and 5b: a schematic view of a monitoring apparatus for monitoring an energy balance having a display,

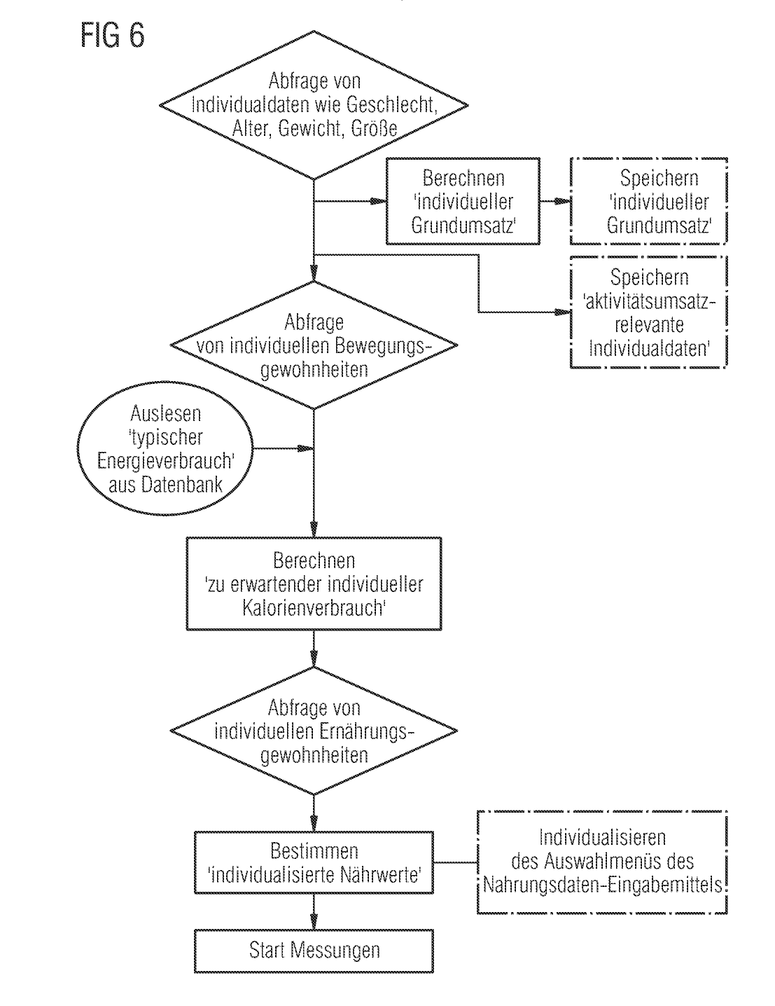

FIG. 6: a flow diagram of a method for individualizing a portable monitoring apparatus,

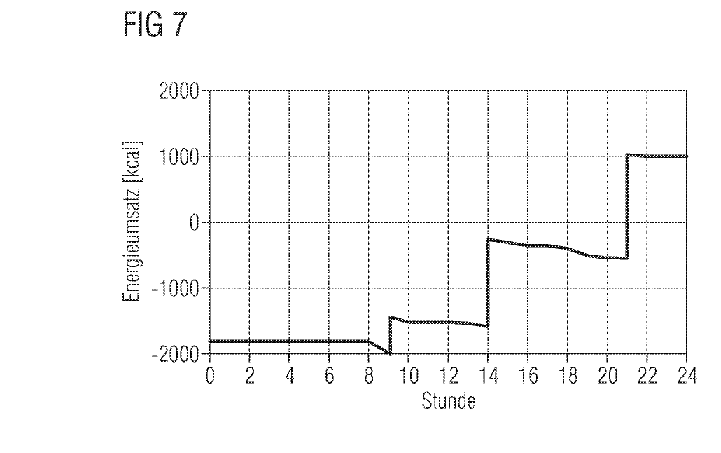

FIG. 7: a diagram with a graph illustrating the energy metabolic balance of a subject over a period of 24 hours, and

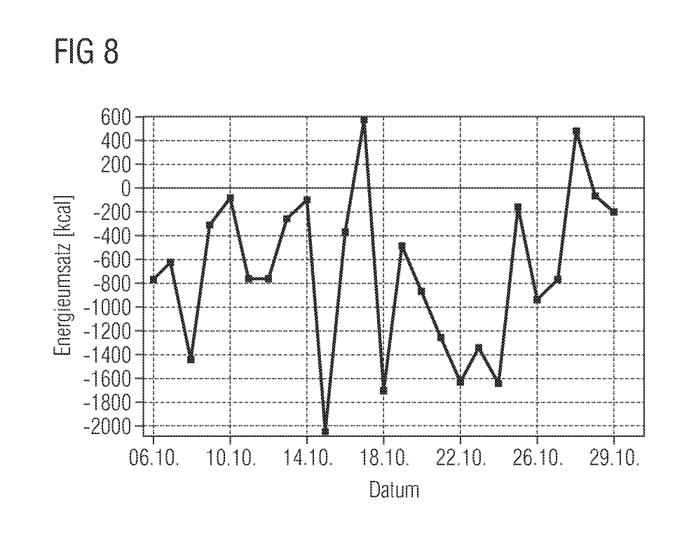

FIG. 8: a diagram with a graph illustrating the energy metabolic balance of a subject over a period of 24 days.

FIG. 1 shows a system 1 having a portable monitoring apparatus 10. The monitoring apparatus 10 comprises a movement data detecting means 56, which may, for example, have a three-axis acceleration sensor. The monitoring apparatus 10 may be carried continuously by a subject and may detect, via the movement data detecting means 56, movement data with regard to movements of the subject and/or movement changes of the subject. Detected movement data may be recorded, e.g. in the monitoring apparatus 10, and may be retrieved for calculation at a later time. It is also conceivable that the movement data are directly incorporated into the calculation of an active metabolic rate without being recorded.

For the purpose of calculating the active metabolic rate a processing unit, denoted as CPU 80, is provided in the monitoring apparatus 10. The CPU 80 serves as the active metabolic rate calculating means 83. The active metabolic rate calculating means 83 may have a signal connection with the movement data detecting means 56 and is designed to calculate an active metabolic rate on the basis of the movement data and specific individual data. Such individual data may be, e.g., the size and/or weight of the subject.

For providing the individual data, an individual data input means 53 and an individual data storing means 61 are arranged in the monitoring apparatus 10. By means of the individual data input means 53, the subject may input further individual data into the monitoring apparatus 10, which may serve to calculate the basal metabolic rate. For calculating the basal metabolic rate, the individual data, which serve to calculate the active metabolic rate, may be used on the one hand. In addition to these data, on the other hand, other individual data, such as age and gender, may be inputted into the system. The basal metabolic rate may be calculated by the CPU 80, comprising for this purpose a basal metabolic rate calculating means 82. For calculating the basal metabolic rate, the basal metabolic rate calculating means 82 may have a signal connection with the individual data storing means 61 and/or with an optionally provided basal metabolic rate storing means, which is not shown in FIG. 1. The basal metabolic rate calculating means 82 may be designed to retrieve the individual data from the individual data storing means 61, calculate the basal metabolic rate on the basis of the individual data and, optionally, store the calculated basal metabolic rate in the basal metabolic rate storing means 63.

According to the embodiment shown in FIG. 1, various individual data may be stored in the individual data storing means 61. The CPU 80 may retrieve these data both for calculating the active metabolic rate and for calculating the basal metabolic rate, and it may calculate the active metabolic rate and the basal metabolic rate on the basis of these retrieved data and the movement data. The active metabolic rate and the basal metabolic rate may be calculated separately, or in one step on the basis of a single algorithm.

Further, the monitoring apparatus 10 as shown comprises a food data input means 52 and a nutritional value data base 71, which may both have a signal connection with the CPU 80, as illustrated in FIG. 1.

By means of the food data input means 52 a subject may enter food data into the monitoring apparatus 10. Corresponding nutritional values may be retrieved, e.g., by the CPU 80, from the nutritional value data base 71. On the basis of the food data, a nutritional value may be determined in the CPU 80. It is further conceivable that several determined nutritional values may be summed up by the CPU 80 to give an overall nutritional value ingested by the subject within a certain period of time, e.g., during one meal. In the embodiment shown, the CPU 80 comprises a nutritional calculating means 84 for this purpose.

On the basis of the calculated nutritional value, the calculated basal metabolic rate and the calculated active metabolic rate an energy balance may be calculated in the CPU 80, for which purpose the CPU 80 also provides an energy balance calculating means 85.

The calculated energy balance may be outputted via a display 51, which provides the function of an energy balance output means. Also, the display 51 may serve as a menu display means of the food data input means 52. According to another embodiment, the food data input means 52 may have its own menu display means, which is arranged separately from the display 51.

According to another embodiment, the basal metabolic rate calculating means 82 could be provided on an external device, such as a computer, so that an externally calculated basal metabolic rate could subsequently be transferred to the monitoring apparatus 10. In this case, the basal metabolic rate would not have to be calculated on the monitoring apparatus 10. In such an embodiment of the system 1, where the basal metabolic rate is not calculated on the monitoring apparatus, the basal metabolic rate may be available in a stored form in the monitoring apparatus 10, so that it may be used as a basis for the calculation described above.

In the embodiment shown in FIG. 1, however, the system does not comprise a computer. Instead, the basal metabolic rate is calculated on demand, if needed, by means of the basal metabolic rate calculating means 82 in the monitoring apparatus 10 without being stored in the monitoring apparatus 10. It is also conceivable to provide in this embodiment a basal metabolic rate storing means as a component of the monitoring apparatus 10.

In the embodiment shown, the system further comprises a balance 41 having a signal connection with a data transfer module 93 of the monitoring apparatus 10. The balance 41 may be a reasonable, but not absolutely necessary supplement of the system 10. Weighing data provided by the balance 41 may be transferred via a signal connection between the balance 41 and the data transfer module 93 to the monitoring apparatus 10, e.g., via a unidirectional data connection. The weighing data may be stored as the subject's weight in the individual data storing means 61, so that in case of a weight change the basal metabolic rate may be determined correctly on the basis of the weighing data.

FIG. 2 shows a second embodiment of the system 1, corresponding substantially with the embodiment in FIG. 1. To avoid unnecessary repetitions, only the differences in comparison with FIG. 1 are described.

In the embodiment shown in FIG. 2, the system 1 has a computer 20. To facilitate a data exchange between the computer 20 and the monitoring apparatus 10, the computer 20 and the monitoring apparatus 10 in this case have two corresponding interface modules. Of the corresponding interface modules, only the interface modules of the monitoring apparatus 10 are shown. The monitoring apparatus 10 has a wireless interface module 94 and a wired interface module 95, so that a signal connection may optionally be established via a wireless signal connection 91 or a wired signal connection 92 between the two devices. The wireless interface module 94 also facilitates a wireless signal connection 91 with the balance 41, which substantially corresponds to the balance described above.

By means of the signal connection between the computer 20 and the monitoring apparatus 10, e.g., data stored in the monitoring apparatus 10 may be transferred to the computer, e.g. in order to be permanently stored there. Thus, the memory of the monitoring apparatus 10 may be designed to be smaller. Moreover, this may prevent data loss. The data transferred to the computer 10 may be processed there and displayed to the subject, as the example in FIGS. 7 and 8 shows with reference to a time-related energy metabolic rate.

FIG. 3 shows a third embodiment of the system 1, for which again only the differences in comparison with FIG. 1 or 2 are described.

In this embodiment the symbolically represented memory chip, comprising the individual data storing means 61, further comprises a food data storing means 62, a basal metabolic rate storing means 63, and a movement information storing means 64. Apart from this memory chip, a second memory chip is provided, comprising the nutritional value data base as well as a movement pattern data base 72. By means of this design, different memory chips may be provided for different tasks. For example, the memory chips 61, 62, 63, and 64 may be arranged as memory areas of a non-permanent, re-writable memory chip. The memory chip on which the data bases 71, 72 are stored, may be arranged as a permanent, non-rewritable memory chip. It is also conceivable that both the storing means and the data bases are placed on one memory chip, or that further memory chips are provided.

The computer 20 has a basal metabolic rate calculating means 82, shown separately for the sake of illustration, which may be provided by a CPU of the computer 20. In this version it is possible, e.g., when putting the monitoring apparatus 10 into operation and individualizing the monitoring apparatus 10, to calculate the basal metabolic rate by the basal metabolic rate calculating means 82, transfer it via a signal connection to the monitoring apparatus 10, and store it there in the basal metabolic rate storing means 63.

The system 10 according to this embodiment shown here may also comprise, in addition to the balance 41, a body fat meter 43 that may be provided as one apparatus together with the balance 41, or as a separate apparatus. Thus, a body fat percentage may be stored as further information together with the individual data. This makes a more exact calculation of the basal metabolic rate possible as the body fat percentage allows more exact conclusions to be drawn about the muscle mass of the subject, which is one of the underlying factors of the basal metabolic rate. Further, the system 10 according to this embodiment comprises a blood sugar meter 42, which may also transfer data to the monitoring apparatus via the wireless signal connection 91. Alternatively or additionally, the system 10 may comprise a blood pressure unit.

Moreover, in this embodiment or in another embodiment allowing for an automatic or manual weight check and correction of the individual data, a weight change calculating means 87 may be provided, whose function, as shown in this embodiment, may be taken over by the CPU 80, or also by the computer 20, or by a data server, as shown in FIG. 4.

The weight change calculating means 87 may be designed to calculate a theoretical weight change that should have occurred over a longer period of time, such as a month, two months, etc., if the calculated data, such as the basal metabolic rate, active metabolic rate and nutritional value were taken to be a correct basis. By means of the balance the actual value of the weight may be compared with the calculated value. A basal metabolic rate correcting means may be designed to correct the basal metabolic rate accordingly if the target value diverges from the actual value. Additionally or alternatively, a nutritional value data correcting means may be provided, e.g., to accordingly correct nutritional value data available in the nutritional value data base in an individualized form.

FIG. 4 shows a third embodiment of the system 10. Again, only the differences as compared to the embodiments described above are explained.

The embodiment of the system 10 shown in FIG. 4 further comprises a central server 30 that may be provided, e.g., on the internet and may establish a wireless signal connection 91 with the other apparatus of the system 10, e.g., via a mobile network. In FIG. 4 a wireless signal connection 91 between the central server 30 and the monitoring apparatus 10 is shown, which may be appropriate if a mobile radio unit is used as a monitoring apparatus 10. The central server 30 may also be connected with the computer 20 via a wired signal connection 92, such as a fixed-network line. In this way data stored in the monitoring apparatus 10 may be transferred to the central server 30. The data may, e.g., be made available to a nutrition coach or physician, who may then give some feedback with instructions to the subject. It is also conceivable that such feedback is issued automatically. Further, it is conceivable that a feedback message is sent automatically in standard cases, and if certain data point to a special case, that the process is automatically transferred to a doctor for consideration.

In the embodiment according to FIG. 4, the CPU 80 of the monitoring apparatus 10 is equipped with a timer, i.e. a time detecting means 81. Data recorded by the monitoring apparatus may be provided with time information supplied by the timer with reference to the exact time and/or duration of certain events.

Finally, the fourth embodiment has an activity input means 55 and a set target input means 54. By means of the activity input means 55, the subject may input activities that were not recorded by the monitoring apparatus into the monitoring apparatus. This may be appropriate, e.g., when the monitoring apparatus was not carried by the subject while engaging in certain sports. By means of the set target input means 54, the subject may enter set target values and may have the monitoring apparatus notify them when these targets have been reached. Further, the monitoring apparatus may comprise an input device for inputting medical parameters, which is not shown in this Figure.

FIGS. 5a and 5b show a schematic view of a monitoring apparatus 10 for monitoring an energy balance.

In FIG. 5a, the display 51 is shown as an energy balance output means. In addition to the balance value as such, the caloric value of food intake, the caloric value of the basal metabolic rate and the caloric value of the active metabolic rate are displayed by the energy balance output means on the display 51.

In FIG. 5b the display 51 is shown as a menu display means of a food data input means. The food data input means further comprises a menu selection means 522, which is arranged as keys in this embodiment. By using these keys, the subject may make a selection from the categorized selection menu shown on the menu display means. For example, the subject may select a certain menu item via the upper two keys and confirm their choice by pressing the key shown on the lower right. In FIG. 5b a second menu layer of a tree structure is shown. At the first menu layer, the menu item ‘main meal’ was selected amongst several meals at choice. At the second menu layer a possible choice amongst ‘small’, ‘medium’ and ‘large’ meals is shown in this embodiment. For each menu item the individualized number of calories is also indicated, which is linked with the relevant menu item in the food data base of the monitoring apparatus.

FIG. 6 shows a flow diagram of an embodiment of a method for individualizing a portable monitoring apparatus.

This method comprises the following steps:
requesting input from a subject with regard to their individual data, such as gender, age, weight, size, etc.,
storing of the individual data, in particular data relating to the active metabolic rate, i.e. the individual data used, together with detected movement data, for calculating the calories expended by physical activities, in the monitoring apparatus,
calculating a basal metabolic rate on the basis of the individual data relating to the basal metabolic rate,
storing the calculated basal metabolic rate in the monitoring apparatus,
requesting input from the subject with regard to their movement habits, in particular with regard to the physical activities the subject engages in every day,
retrieving a typical energy expenditure, e.g. a specific caloric expenditure, corresponding to the physical activities, from a data base,
calculating the subject's expected caloric expenditure on the basis of the retrieved energy expenditure, particularly on the basis of the activity-related individual data,
requesting input from the subject with regard to their dietary habits,
determining individualized nutritional values with regard to individual meals, on the basis of the requested dietary habits, and, optionally, on the basis of the basal metabolic rate and/or the caloric expenditure, and
individualizing the selection menu of a food data input means on the basis of the determined nutritional values.

Upon completing individualization, the monitoring apparatus may be put in operation, and the measurements may be started.

### LIST OF REFERENCE NUMBERS

1 system
10 monitoring apparatus
20 computer
30 central server
41 balance
42 blood sugar meter
43 body-fat meter
51 display
52 food data input means
522 menu selecting means
53 individual data input means
54 set target input means
55 activity input means
56 movement data detecting means
61 individual data storing means
62 food data storing means
63 basal metabolic rate storing means
64 movement information storing means
71 nutritional value data base
72 movement pattern data base
80 CPU
81 time detecting means
82 basal metabolic rate calculating means
83 active metabolic rate calculating means
84 nutritional value calculating means
85 energy balance calculating means
87 weight change calculating means
91 wireless signal connection
92 wired signal connection
93 data transfer module
94 wireless interface module
95 wired interface module

1. System for monitoring an energy balance of a subject, comprising:
an individual data input means designed to input subject-related individual data into the system,
an individual data storing means designed to store the inputted individual data in the system,
a basal metabolic rate calculating means designed to calculate a subject-related basal metabolic rate on the basis of the individual data and provide it in the system,
a food data input means designed to facilitate the input of food data by means of individualized categories into the system,
a nutritional value data base having nutritional values assigned to the food data,
a nutritional value calculating means designed to calculate an overall nutritional value on the basis of the inputted food data and the nutritional values assigned thereto in the nutritional data base,
a movement data detecting means designed to be operatively arranged on the subject and to detect movements and/or movement changes of the subject, and to provide corresponding movement data,
an active metabolic rate calculating means designed to calculate an active metabolic rate on the basis of the detected movement data,
an energy balance calculating means designed to calculate an energy balance on the basis of the active metabolic rate, the basal metabolic rate and the nutritional value, and
an energy balance output means for outputting the determined energy balance.

2. System according to claim 1, wherein the basal metabolic rate calculating means has a signal connection with a basal metabolic rate storing means designed to store the basal metabolic rate calculated by the basal metabolic rate calculation means.

3. System according to claim 2, wherein the basal metabolic rate storage means has a signal connection with the energy balance calculating means, wherein the energy balance calculating means is designed to retrieve the basal metabolic rate stored in the basal metabolic rate storing means.

4. System according to claim 1, wherein no basal metabolic rate storing means is provided and wherein the basal metabolic rate calculating means has a signal connection with the individual data storing means, wherein the basal metabolic rate calculating means is designed to calculate the basal metabolic rate on the basis of the individual data stored in the system and to provide the calculated basal metabolic rate to the energy balance calculating means.

5. System according claim 1, wherein the food data input means comprises a menu display means and a menu selection means, wherein the menu display means is designed to display to the subject a categorized selection menu, wherein the selection menu preferably has a tree structure showing several menu layers and several items per menu layer and is designed to enable the user to input the food data via the tree structure.

6. System according to claim 5, wherein the tree structure of the categorized selection menu does not have more than 10 items per menu layer and/or wherein the tree structure does not have more than 5 menu layers.

7. System according to claim 5, wherein the food data input means is designed to enable the subject to input concrete food data in addition to the categorized selection menu.

8. System according to claim 5, wherein the food data input means is designed to be individualized by the subject.

9. System according to claim 8, wherein the possibility of individualization of the food data input means relates to the limitation of the number of menu layers, the limitation of the number of menu items per menu layer, the name of menu items, and/or the nutritional values assigned to certain menu items.

10. System according to claim 1, wherein the system has a food data storing means designed to store food data inputted via the food data input means and/or assigned nutritional values in the system.

11. System according to claim 1, wherein the system has a movement pattern data base with movement patterns stored in the movement pattern data base that may be linked with the detected movement data.

12. System according to claim 1, wherein the system has a movement information storing means designed to store movement data provided via the movement data detecting means and/or movement patterns linked with the movement data in the system.

13. System according to claim 1 having a time detecting means designed to link a basal metabolic rate and/or a food data input and/or movement data and/or a movement pattern with time data.

14. System according to claim 1, comprising a distance calculating means.

15. System according to claim 1, comprising a sensing means designed to detect a physiological parameter of a subject.

16. System according to claim 1, comprising a set target input means designed to enable the subject to input a set target.

17. System according to claim 1, comprising an activity input means designed to enable a subject to input physical activities.

18. System according to claim 1, comprising a weight change calculating means, a weight comparing means and a basal metabolic rate correcting means.

19. System according to claim 1, comprising a portable monitoring apparatus, suitable for being carried by a subject, wherein the monitoring apparatus comprises at least one of the following components:
the individual data storing means,
the food data input means,
the nutritional value data base,
the nutritional value calculating means,
the movement data detecting means,
the active metabolic rate calculating means,
the energy balance calculating means, and
the energy balance output means.

20. System according to claim 19, wherein the system further comprises a computer and/or a balance and/or a blood sugar meter and/or a body fat meter and/or a central server.

21. System according to claim 20, wherein a signal connection between the monitoring apparatus and the computer and/or between the monitoring apparatus and the balance and/or between the balance and the computer is arranged as a wired signal connection or as a wireless signal connection.

22. System according to claim 1, arranged as a portable monitoring apparatus suitable for being carried by a subject.

23. Computer program product, comprising program parts, which, when loaded onto a processor and executed, are designed for monitoring the energy balance of a subject, for executing the following steps:
requesting of individual data,
calculating a basal metabolic rate on the basis of individual data related to the basal metabolic rate,
requesting of food data,
determining a nutritional value on the basis of the requested food data,
detecting of movement data,
calculating an active metabolic rate on the basis of the movement data and the individual data related to the active metabolic rate,
calculating an energy balance on the basis of the basal metabolic rate, the active metabolic rate and the nutritional value,
outputting of the calculated energy balance.
